(19) United States
(12) Patent Application Publication
(10) Pub. No.: US 2015/0238606 A1
(43) Pub. Date: Aug. 27, 2015

(54) PHARMACEUTICAL COMPOSITION CONTAINING LULICONAZOLE

(71) Applicants: POLA PHARMA INC., Tokyo (JP); NIHON NOHYAKU CO., LTD., Tokyo (JP)

(72) Inventors: Takaaki Masuda, Kanagawa (JP); Hirokazu Kobayashi, Kanagawa (JP)

(73) Assignees: POLA PHARMA INC.,, Shinagawa,-ku, Tokyo (JP); NIHON NOHYAKU CO., LTD.,, Chuo-ku, Tokyo (JP)

(21) Appl. No.: 14/427,890

(22) PCT Filed: Aug. 28, 2013
(86) PCT No.: PCT/JP2013/073731

§ 371 Date: Mar. 12, 2015

(30) Foreign Application Priority Data
Sep. 14, 2012 (JP) 2012-203293

(51) Int. Cl. A61K 47/14; A61K 47/22; A61K 47/34; A61K 47/08; A61K 31/4178; A61K 47/10

(52) U.S. Cl. CPC A61K 47/14; A61K 31/4178; A61K 47/10; A61K 47/34; A61K 47/08; A61K 47/22

(57) ABSTRACT

Means for controlling the formation amount of a formed amide form in relation to a pharmaceutical composition containing luliconazole is provided. Disclosed is a pharmaceutical composition containing 1) luliconazole and 2) one component or two or more components selected from carboxylic acid and derivative thereof, ketone, phosphoric acid and derivative thereof, local anesthetic, antihistamine, and POE-based nonionic surfactant; wherein a content of an amide derivative of luliconazole is not more than 0.2% by mass with respect to a charged amount of luliconazole after storage at 60° C. for 3 weeks or at 40° C. for 6 months.

### TECHNICAL FIELD

The present invention relates to a pharmaceutical composition. In particular, the present invention relates to a pharmaceutical composition containing luliconazole in which production or formation of a luliconazole analog is suppressed.

### BACKGROUND ART

Luliconazole is an antifungal agent which is excellent in the action on fungi. At present, luliconazole is widely used as a pharmaceutical or medicine for tinea pedis and tinea corporis, and it is going to be applied also for the action on tinea unguium. In relation to the pharmaceutical preparation (medicament preparation) of luliconazole, it is known as problems which should be solved that luliconazole is converted to stereoisomers such as the SE isomer and the Z isomer, and that the crystallization of luliconazole is caused immediately after the application (see, for example, Patent Documents 1 to 6).

### PRECEDING TECHNICAL DOCUMENTS

### Patent Documents

Patent Document 1: WO2007/102241;
Patent Document 2: WO2007/102242;
Patent Document 3: WO2007/102243;
Patent Document 4: WO2009/031642;
Patent Document 5: WO2009/031643;
Patent Document 6: WO2009/031644.

### SUMMARY OF THE INVENTION

### Technical Problem

When researches are promoted about the dosage form preparation of this substance, it has been revealed that an analog, which was not known during the development process of this substance, occasionally appears during the storage period by performing the storage test under a severe condition (storage test at 60° C. for 3 weeks) or under an accelerated condition (storage test at 40° C. for 6 months) in relation to the pharmaceutical preparation. At present, it has been revealed that this analog is not originally contained in the active ingredient, and the analog is produced or formed for the first time when the pharmaceutical preparation is prepared and subjected to the storage test. The situation of production or formation of this analog differs depending on the type of the selected solvent. As a result of the confirmation of the structure, it has been revealed that the analog is an amide form in which the nitrile group of luliconazole is hydrolyzed and converted into the amide group. As described above, the amide form of luliconazole does not result from the active ingredient. Therefore, the amide form of luliconazole is produced or formed for the first time by preparing the pharmaceutical preparation by accidentally adding the component which easily facilitates the formation of the amide form and performing the accelerated test or the severe test. Therefore, it is affirmed that any luliconazole pharmaceutical preparation, for which it has been shown that the amide form of luliconazole is contained, is not present in the world until now in the present circumstances.

In the development process of luliconazole, the amide derivative, which is represented by Chemical Formula (2), is not known at all. It is also unknown at all that the presence or absence of the formation of this substance is an important index in relation to the selection of the solvent for the pharmaceutical preparation. Further, the amide derivative represented by Chemical Formula (2) is a novel substance having been not described in any literature. In general, it is known that nitrile is subjected to the water addition reaction caused by acid or alkali and nitrile is converted into amide. Therefore, it is considered that the substance as described above is formed by the addition of water to the nitrile group of luliconazole. However, it is hardly speculated in ordinary cases that such a compound is produced or formed under the storage condition depending on the type of the solvent. It is also unknown at all and it is also impossible to predict that the production of the amide form as described above can be avoided by combining any component which is neither acidic nor alkaline. Further, any relationship is also unknown at all between the nitrile group and isopropyl myristate, middle-chain fatty acid triglyceride, triacetin, triethyl citrate, acetone, methyl ethyl ketone, POE fatty acid ester, POE alkyl (alkenyl) ether, sorbitan fatty acid ester, POE sorbitan fatty acid ester, POE hydrogenated castor oil, dibasic acid ester, hydroxyethylidene diphosphonic acid, and ethylene glycol salicylate. Such an exemplary case is scarcely known until now that the production or formation of the amide form is facilitated or suppressed depending on the type of the solvent to be used.

In the case of a preparation of luliconazole for external use in which the total administration amount per day is not more than 1 g in relation to the preparation for external use, it is determined by the Pharmaceutical Affairs Law whether or not the follow-up (tracing) survey to be performed later on is required, depending on whether the formation amount of analog is above 0.2% or not more than 0.2%. According to this fact, it is extremely significant that the number of analogs as application items can be decreased by suppressing the formation amount of any specified analog substance to be not more than 0.2%. It is no exaggeration to say that the appearance of such means is waited for. Further, in relation to the preparation for external use containing luliconazole, it is affirmed that a pharmaceutical preparation, in which the formation amount of the amide form is not more than 0.2% with respect to the active ingredient under a severe condition of the storage at 60° C. for 3 weeks although the pharmaceutical preparation contains any component or ingredient to facilitate the formation of the amide form, is a useful pharmaceutical preparation as the pharmaceutical preparation of luliconazole.

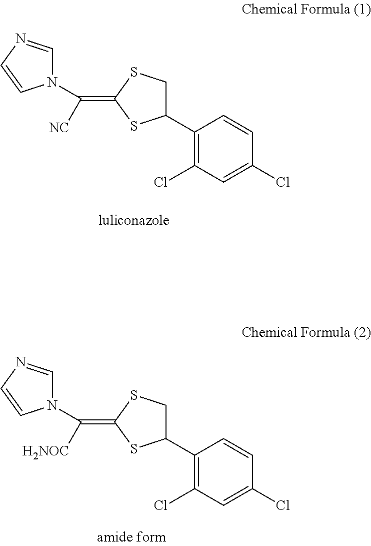 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

The present invention has been made in the circumstances as described above, an object of which is to provide means for controlling the formation amount of the amide form occasionally produced or formed depending on the combination of formulation components, in other words, means for stabilizing the nitrile group of luliconazole.

### Solution to Problem

Taking the foregoing circumstances into consideration, the present inventors have repeatedly and diligently performed researches and efforts in order to seek for means for controlling the formation amount of the amide form occasionally produced or formed depending on the combination of formulation components. As a result, it has been found out that such an action exists in a component selected from carboxylic acid and derivative thereof, ketone, phosphoric acid and derivative thereof, local anesthetic, antihistamine, and POE-based nonionic surfactant. Thus, the invention has been completed. That is, the present invention is as follows.

<1> A pharmaceutical composition containing 1) luliconazole represented by Chemical Formula (1) and 2) one component or two or more components selected from carboxylic acid and derivative thereof, ketone, phosphoric acid and derivative thereof, local anesthetic, antihistamine, and POE-based nonionic surfactant, wherein:

a content of an amide form represented by Chemical Formula (2) is not more than 0.2% by mass with respect to a charged amount of luliconazole after storage at 60° C. for 3 weeks or at 40° C. for 6 months:

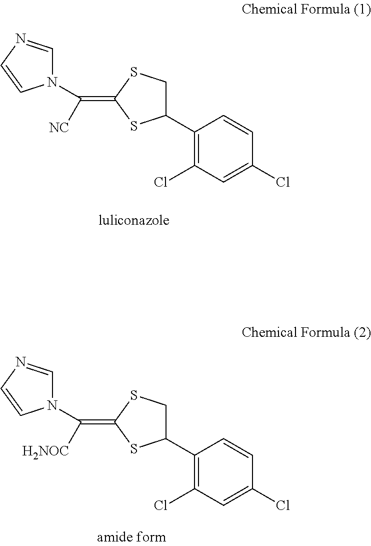 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

<2> The pharmaceutical composition as defined in <1>, wherein the component, which is selected from carboxylic acid and derivative thereof, ketone, phosphoric acid and derivative thereof, local anesthetic, antihistamine, and POE-based nonionic surfactant, is selected from the following group (A):

### (A)

carboxylic acid and derivative thereof selected from: isopropyl myristate, sorbitan fatty acid ester, middle-chain fatty acid triglyceride, triacetin, triethyl citrate, dibasic acid ester, and ethylene glycol salicylate;

ketone selected from: acetone, methyl ethyl ketone;

phosphoric acid and derivative thereof selected from: hydroxyethylidene diphosphonic acid;

local anesthetic selected from: lidocaine and salt thereof;

antihistamine selected from: diphenhydramine and salt thereof;

POE-based nonionic surfactant selected from: POE fatty acid ester, POE alkyl (alkenyl) ether, POE sorbitan fatty acid ester, POE hydrogenated castor oil.

<3> The pharmaceutical composition as defined in <1> or <2>, further containing one component or two or more components selected from polyhydric alcohol, middle-chain, long-chain, or cyclic monohydric alcohol, and pyrrolidone and derivative thereof.

<4> The pharmaceutical composition as defined in <3>, wherein polyhydric alcohol, middle-chain, long-chain, or cyclic monohydric alcohol, and pyrrolidone and derivative thereof are selected from the following group (B):

### (B)

polyhydric alcohol selected from: 1,3-butanediol, polyethylene glycol, propylene glycol, polypropylene glycol, and glycerol;

middle-chain, long-chain, or cyclic monohydric alcohol selected from: benzyl alcohol, oleyl alcohol, and isostearyl alcohol;

pyrrolidone and derivative thereof selected from: pyrrolidone and derivative thereof.

<5> A pharmaceutical composition containing 1) luliconazole represented by Chemical Formula (1) and 2) one component or two or more components selected from polyhydric alcohol, middle-chain, long-chain, or cyclic monohydric alcohol, and pyrrolidone and derivative thereof, by less than 1% by mass, wherein:

a content of an amide form represented by Chemical Formula (2) is not more than 0.2% by mass with respect to a charged amount of luliconazole after storage at 60° C. for 3 weeks or at 40° C. for 6 months:

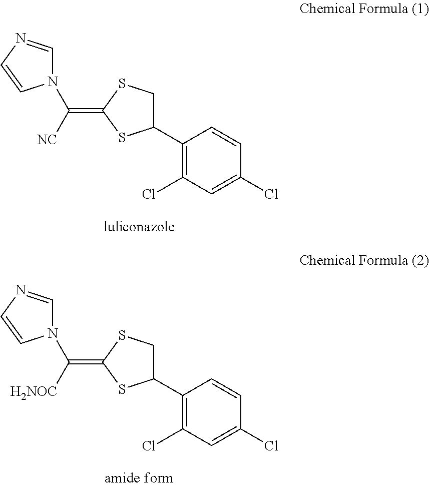 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

<6> The pharmaceutical composition as defined in <5>, wherein the one component or the two or more components selected from polyhydric alcohol, middle-chain, long-chain, or cyclic monohydric alcohol, and pyrrolidone and derivative thereof is/are selected from the following group (B):

### (B)

polyhydric alcohol selected from: 1,3-butanediol, polyethylene glycol, propylene glycol, polypropylene glycol, and glycerol;

middle-chain, long-chain, or cyclic monohydric alcohol selected from: benzyl alcohol, oleyl alcohol, and isostearyl alcohol;

pyrrolidone and derivative thereof selected from: pyrrolidone and derivative thereof.

<7> The pharmaceutical composition as defined in any one of <1> to <6>, wherein the pharmaceutical composition is a liquid agent or a cream agent.

<8> A conversion-suppressing agent for suppressing conversion of luliconazole represented by Chemical Formula (1) into an amide form represented by Chemical Formula (2) under a storage condition at 60° C. for 3 weeks or at 40° C. for 6 months, the conversion-suppressing agent consisting of one component or two or more components selected from carboxylic acid and derivative thereof, ketone, phosphoric acid and derivative thereof, local anesthetic, antihistamine, and POE-based nonionic surfactant:

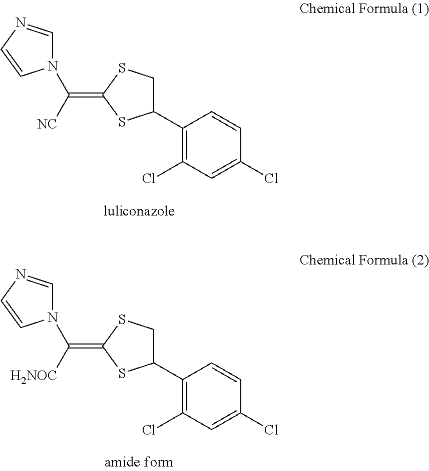 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

<9> A method for producing a pharmaceutical composition containing luliconazole as a principal agent, the method comprising:

a step of preparing a pharmaceutical preparation by allowing 1) luliconazole represented by Chemical Formula (1) to be contained together with 2) one component or two or more components selected from carboxylic acid and derivative thereof, ketone, phosphoric acid and derivative thereof, local anesthetic, antihistamine, and POE-based nonionic surfactant and 3) one component or two or more components selected from polyhydric alcohol, middle-chain, long-chain, or cyclic monohydric alcohol, and pyrrolidone and derivative thereof; and

a step of confirming that an amount of an amide form is not more than 0.2% by mass of a charged amount of luliconazole by performing a storage test under a severe condition (60° C. for 3 weeks) or an accelerated condition (40° C. for 6 months):

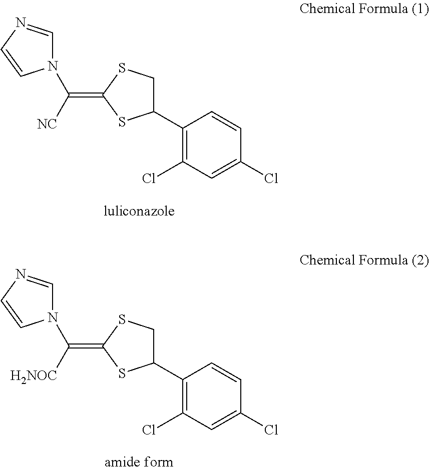 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

<10> A pharmaceutical composition comprising luliconazole as a principal agent produced by the method as defined in <9>.

### Advantageous Effects of Invention

According to the present invention, it is possible to provide means for controlling the formation amount of the amide form occasionally produced or formed depending on the combination of formulation components.

### DESCRIPTION OF EMBODIMENTS

### <1> Pharmaceutical Composition of the Present Invention

The pharmaceutical composition of the present invention is a pharmaceutical composition containing luliconazole, which resides in such a pharmaceutical preparation that the formation amount of the amide form (Chemical Formula (2)) produced or formed from luliconazole in the production process or the storage process is suppressed. Specifically, the pharmaceutical composition of the present invention has the following feature. The formation amount of the amide form is not more than 0.2% by mass with respect to the charged amount (blending amount) of luliconazole after the storage at 60° C. for 3 weeks or under the storage condition at 40° C. for 6 months. That is, the formation amount of the amide form is not more than 0.002% by mass with respect to the total amount of the pharmaceutical preparation, in the case of the pharmaceutical preparation in which the content of luliconazole is 1% by mass. More preferably, the formation amount of the amide form is not more than 0.1% by mass, and the formation amount of the amide form is not more than 0.001% by mass with respect to the pharmaceutical preparation in the case of the pharmaceutical preparation in which the content of luliconazole is 1% by mass.

The preferred content of luliconazole in the pharmaceutical composition of the present invention is 0.1 to 20% by mass. The content of luliconazole is more preferably 0.5 to 15% by mass, and much more preferably 1 to 10% by mass.

The pharmaceutical composition of the present invention is preferably exemplified by preparations for external use including, for example, liquid agent, cream agent, gel, foam, spray agent, and ointment. In order to prepare the pharmaceutical composition having the property as described above, the following procedure is preferably exemplified. That is, the component which easily forms or produces the amide form and the component which suppresses the production or formation of the amide form are distinguished or discriminated from the components for preparing the pharmaceutical preparation, and they are selected and classified into groups. When at least the component, which easily forms or produces the amide form, is used as the pharmaceutical preparation component, the pharmaceutical preparation is designed so that the component, which suppresses the production or formation of the amide form, is contained.

It is preferable that the component, which suppresses the formation of the amide form, is contained actively or positively. From such a viewpoint, the pharmaceutical composition of the present invention contains, as the essential component, the component which suppresses the production or formation of the amide form and which is selected from carboxylic acid and derivative thereof, ketone, phosphoric acid and derivative thereof, local anesthetic, antihistamine, and POE-based nonionic surfactant. One component or two or more components selected therefrom may be contained.

The component, which easily produces or forms the amide form, can be exemplified by polyhydric alcohol, middle-chain, long-chain, or cyclic monohydric alcohol, and pyrrolidone and derivative thereof. When the component as described above is contained, it is preferable that the component, which hardly produces or forms the amide form as described above, is simultaneously contained. It is possible that one component or two or more components selected therefrom is/are contained.

Among the pharmaceutical preparation as the pharmaceutical composition obtained by combining the components as described above, in case that it is confirmed that the formation amount of the amide form is not more than 2% by mass with respect to the charged amount of luliconazole after the storage at 40° C. for 6 months or under the storage condition at 60° C. for 3 weeks, i.e., the content of luliconazole is 1% by mass and the formation amount is not more than 0.002% by mass with respect to the total amount of the pharmaceutical preparation, the concerning pharmaceutical preparation is the pharmaceutical composition of the present invention. As a result of the confirmation by the present inventors, the storage condition at 60° C. for 3 weeks and the storage condition at 40° C. for 6 months are greatly correlated with each other in many parts, and it is possible to use any one of the both conditions. That is, as for the results of the storage condition at 60° C. for 3 weeks and the storage condition at 40° C. for 6 months, one of the results can be interpreted as the other result. However, it is preferable to adopt the condition at 60° C. for 3 weeks, because the evaluation can be performed in a short period of time.

The pharmaceutical composition of the present invention, which contains luliconazole as the principal agent, can be produced by allowing luliconazole represented by Chemical Formula (1) to be contained together with one component or two or more components selected from carboxylic acid and derivative thereof, ketone, phosphoric acid and derivative thereof, local anesthetic, antihistamine, and POE-based nonionic surfactant to prepare a pharmaceutical preparation; and performing a storage test under a severe condition (60° C. for 3 weeks) or an accelerated condition (40° C. for 6 months) to confirm that an amount of an amide form is not more than 0.2% by mass of a charged amount of luliconazole. Further, when the pharmaceutical composition contains one component or two or more components selected from polyhydric alcohol, middle-chain, long-chain, or cyclic monohydric alcohol, and pyrrolidone and derivative thereof, the pharmaceutical composition can be also produced by preparing a pharmaceutical preparation in the same manner as described above and performing a storage test under a severe condition (60° C. for 3 weeks) or an accelerated condition (40° C. for 6 months) to confirm that an amount of an amide form is not more than 0.2% by mass of a charged amount of luliconazole.

When the amide form as described above is produced, the production can be performed by treating luliconazole together with water in the presence of a metal catalyst such as copper, iridium, alumina, hydroxyapatite or the like. Alternatively, the amide form can be also obtained by allowing acid or alkali to act on luliconazole in water-containing ethanol. The amide form thus obtained can be purified, for example, by means of chromatography such as silica gel column chromatography, octadecyl-modified silica gel column chromatography or the like or by means of recrystallization, for example, from mixture liquid of ethyl acetate-normal hexane, ethanol, isopropanol or the like. The obtained amide form can be used as a standard substance (standard reference material), which can be used as an index for analog of luliconazole in the method for producing the pharmaceutical composition containing luliconazole as the principal agent according to the present invention. The characteristic values of the amide form are as follow.

¹H-NMR (CDCl₃, ppm): 3.617 (dd, 1H), 3.639 (dd, 1H), 5.554 (dd, 1H), 6.993 (s, 1H), 7.231 to 7.311 (m, 2H), 7.447 to 7.664 (m, 3H)

m.p.: 238 to 244° C.

The amide form as described above can be also detected and quantitatively measured by HPLC. When analogs of luliconazole are confirmed, a chiral normal phase column is used in many cases in order to distinguish isomers such as SE isomer and Z isomer. However, the compound represented by Chemical Formula (1) is hardly detected under the elution condition of the chiral normal phase column. Therefore, it is preferable to perform the investigation under a condition in which a reverse phase column is used by using cation-capturing counter ion such as alkyl sulfonate or the like. The analysis condition as described above can be preferably exemplified by the following. Under this condition, the major analogs such as the SE isomer, the Z isomer or the like can be also detected together with luliconazole. The following condition is especially preferable of the conditions as described above.

Column: ODS-2 4.6×150 mm, column temperature: 40° C., mobile phase: 0.15% sodium undecane-1-sulfonate mixture liquid (water/acetonitrile/acetic acid (100) (50:49:1, v/v/v)) solution, flow rate: 1.0 mL/min., detection: 295 nm.

### <2> Component for Suppressing Formation of Amide Form

The component, which suppresses the production or formation of the amide form, is exemplified by the component selected from carboxylic acid and derivative thereof, ketone, phosphoric acid and derivative thereof, local anesthetic, antihistamine, and POE-based nonionic surfactant.

Specifically, as for carboxylic acid, it is possible to preferably exemplify, for example, hydroxy acid such as lactic acid, citric acid, and tartaric acid; aliphatic carboxylic acid such as formic acid, carbonic acid, acetic acid, and fatty acid; and aromatic carboxylic acid such as benzoic acid and salicylic acid. As for the derivative, it is possible to preferably exemplify salt or ester. As for carboxylic acid salt, for example, it is possible to preferably exemplify, for example, alkali metal salt such as sodium hydrogencarbonate, sodium dihydrogen citrate, sodium tartrate or the like. As for carboxylic acid ester, it is possible to preferably exemplify isopropyl myristate, cetyl isooctanate, octyl dodecyl oleate, glycerol monostearate, triethyl citrate, ethylene glycol salicylate, sorbitan fatty acid ester (for example, sorbitan monostearate), or dibasic acid ester such as diisopropyl adipate, diethyl adipate, diethyl sebacate, ethylene carbonate, propylene carbonate or the like, or triglyceride such as middle-chain fatty acid (number of carbon atoms: 8 to 12) glycerol triester, olive oil, isostearic acid glycerol triester, triacetin or the like. Among them, it is possible to preferably exemplify, for example, diisopropyl adipate.

As for ketone, it is possible to preferably exemplify acetone and methyl ethyl ketone.

As for POE-based nonionic surfactant, it is possible to preferably exemplify POE fatty acid ester, POE alkyl (alkenyl) ether, POE sorbitan fatty acid ester, POE hydrogenated castor oil.

As for POE fatty acid ester, it is possible to preferably exemplify, for example, POE oleic acid ester, POE stearic acid ester, POE isostearic acid ester, POE myristic acid ester, and POE lauric acid ester. As for POE alkyl (alkenyl) ether, it is possible to preferably exemplify, for example, diethylene glycol monoethyl ether, diethylene glycol diethyl ether, triethylene glycol monoethyl ether, POE lauryl ether, POE cetyl ether, POE stearyl ether, POE isostearyl ether, POE oleyl ether, and POE behenyl ether. As for POE sorbitan fatty acid ester, for example, it is possible to preferably exemplify POE sorbitan oleic acid ester, POE sorbitan stearic acid ester, and POE sorbitan isostearic acid ester.

In the case of the POE-based nonionic surfactant, the number of moles of addition of the polyoxyethylene group is preferably 10 to 40 and more preferably 15 to 30.

As for phosphoric acid and derivative thereof, for example, it is possible to preferably exemplify, for example, phosphoric acid, pharmaceutically acceptable phosphoric acid salt, and hydroxyethylidene diphosphonic acid.

As for local anesthetic, it is preferable to adopt any amide type local anesthetic. It is possible to preferably exemplify lidocaine and pharmaceutically acceptable salt thereof.

As for antihistamine, it is preferable to adopt diphenhydramine-based antihistamine and chlorpheniramine-based antihistamine. It is possible to preferably exemplify diphenhydramine and pharmaceutically acceptable salt thereof and chlorpheniramine and pharmaceutically acceptable salt thereof.

When the component, which suppresses the formation of the amide form as described above, is contained by not less than 1% by mass, the contribution to the suppression of the formation of the amide form is confirmed. Therefore, it is possible to preferably exemplify a case in which the component for suppressing the formation of the amide form is contained by not less than 1% by mass, and it is possible to more preferably exemplify a case in which the component is contained by not less than 5% by mass. In view of the restriction concerning the physical property in relation to the formulation, it is preferable that the component is contained by not more than 30% by mass, and it is especially preferable that the component is contained by not more than 15% by mass.

### <3> Component for Facilitating Formation of Amide Form

The component for the pharmaceutical preparation, which facilitates the production or formation of the amide form, can be preferably exemplified by polyhydric alcohol, middle-chain, long-chain, or cyclic monohydric alcohol, and pyrrolidone and derivative thereof.

As for polyhydric alcohol, it is possible to exemplify polyhydric alcohol having a number of carbon atoms of 3 to 1,000. It is possible to preferably exemplify 1,3-butanediol, polyethylene glycol, propylene glycol, polypropylene glycol, and glycerol. As for middle-chain, long-chain, or cyclic alcohol, it is allowable to use either aliphatic alcohol or aromatic alcohol. As for aliphatic alcohol, it is possible to exemplify alcohol having a number of carbon atoms of 8 to 30. It is possible to preferably exemplify, for example, cetanol (cetyl alcohol), lauryl alcohol, oleyl alcohol, isostearyl alcohol, cetostearyl alcohol, stearyl alcohol, and behenyl alcohol. As for aromatic alcohol, it is possible to preferably exemplify, for example, benzyl alcohol and phenethyl alcohol.

As for pyrrolidone and derivative thereof, it is possible to preferably exemplify, for example, pyrrolidone carboxylic acid, N-alkyl-2-pyrrolidone such as N-methyl-2-pyrrolidone, N-ethyl-2-pyrrolidone and N-propyl-2-pyrrolidone.

When the component as described above is contained by not less than 1% by mass, the contribution to the formation of the amide form is confirmed. Therefore, when the component as described above is contained by not less than 1% by mass, it is possible to preferably exemplify that the component for suppressing the formation of the amide form is contained together. When the component as described above is contained by not less than 5% by mass, it is possible to more preferably exemplify that the component for suppressing the formation of the amide form is contained together. Even if the necessity arises in view of the formulation, then it is preferable that the component as described above is contained by not more than at least 30% by mass, and it is especially preferable that the component as described above is contained by not more than 15% by mass. It is required that the content should be decreased as much as possible in relation to the component which facilitates the formation of the amide form as described above. However, the component as described above is indispensable for the preparation of the pharmaceutical preparation in many cases, for example, in order to solubilize the active component or ingredient. In such a situation, it is essential that the component, which suppresses the formation of the amide form as described above, should be contained. Depending on, for example, the type of the component, it is preferable that the component, which suppresses the formation of the amide form as described above, is contained approximately by at least the same mass with respect to the component which facilitates the formation of the amide form. Among the components as described above, benzyl alcohol is apt to be generally used, because benzyl alcohol is excellent in the solubilizing performance. However, in the case of luliconazole, benzyl alcohol is a great factor to form the amide form. Therefore, when this component is used as the solubilizing agent, it is preferable to avoid the simultaneous use together with other amide form formation-facilitating component such as cetostearyl alcohol, isostearyl alcohol, propylene glycol or the like. In particular, it is not preferable to combine this component with three or more components selected from other amide form formation-facilitating components, such as a combination of isostearyl alcohol, cetostearyl alcohol, and propylene glycol, because the risk to form the amide form is increased. In such a situation, it is preferable to combine any component which facilitates the suppression of the amide form formation in the same manner as described above. On the other hand, the component such as polyethylene glycol, which facilitates the amide form formation, does not have the action to facilitate the amide form formation so much as compared with benzyl alcohol. Therefore, it is unnecessary for this component to excessively combine and use the amide form formation-suppressing agent(s) for only the concerning purpose, as exemplified, for example, by a combination of middle-chain fatty acid triglyceride, phosphoric acid, and methyl ethyl ketone. Such excessive use is not preferable, because the degree of freedom of the formulation is decreased. Further, it is affirmed that any pharmaceutical preparation, in which the formation amount of the amide form is extremely small under the severe condition at 60° C. for 3 weeks or the accelerated condition at 40° C. for 6 months, specifically the formation amount of the amide form is not more than 0.1% by mass with respect to the total blending amount of luliconazole, although the component for facilitating the production of the amide form is contained as described above, is also an extremely useful pharmaceutical preparation, even when the mechanism of the suppression is unknown.

### <4> Arbitrary Component

The pharmaceutical composition of the present invention can contain an arbitrary component to be usually contained by the pharmaceutical composition. As for the arbitrary component as described above, it is possible to preferably exemplify, for example, hydrocarbons including, for example, Vaseline, microcrystalline wax, and liquid paraffin; silicones including, for example, dimethicone and cyclomethicone; esters including, for example, spermaceti and Japan tallow; triglycerides including, for example, olive oil, beef tallow, and coconut oil; nonionic surfactants not belonging to the essential components including, for example, stearic acid monoglyceride, oleic acid monoglyceride, and POE stearic acid monoglyceride; anionic surfactants including, for example, sodium lauryl sulfate and POE sodium lauryl sulfate; fatty acids including, for example, stearic acid, oleic acid, lauric acid, palmitic acid, and myristic acid; antioxidants including, for example, BHT, BHA, and tocopherol; coloring agents; lubricants; and taste/odor-correcting agents. The pharmaceutical composition of the present invention can be produced by treating the components as described above in accordance with an ordinary method and confirming that the production or formation of the amide form is suppressed in accordance with the storage test.

The pharmaceutical composition of the present invention is preferably used to treat or cure the disease caused by any fungus or prevent the deterioration of the disease by utilizing the characteristic of luliconazole. The disease caused by any fungus can be exemplified by tinea pedis such as athlete's foot, tinea corporis such as candidiasis and tinea versicolor, and trichophytosis of hard keratin portion such as tinea unguium. It is especially preferable to use the pharmaceutical composition of the present invention for treating the disease of the hard keratin portion such as tinea unguium, because the effect thereof is remarkable. The effect of the pharmaceutical composition of the present invention is expressed on the nail especially preferably. However, the effect is also exerted on any ordinary dermatomycosis. Therefore, the pharmaceutical composition, which is directed to the dermatomycosis and which fulfills the construction of the present invention, also belongs to the technical scope of the present invention. The dermatomycosis as described above can be exemplified, for example, by the tinea pedis and the trichophytosis of the propagation in horny substance type appearing, for example, in the heel and being included in the tinea pedis. As for the dermatomycosis described above, it is preferable to make the application to the trichophytosis of the propagation in horny substance type on which any ordinary agent or drug hardly exerts the effect, because the effect of the present invention remarkably arises.

The mode of use can be appropriately selected while considering, for example, the body weight, the age, the sexuality, and the symptoms or condition of the patient. However, in the case of an adult, it is preferable to administer luliconazole in an amount of 0.01 to 1 g per day in ordinary cases. Reference can be made to the amount of use of luliconazole ordinarily used for the disease caused by any fungus.

For example, in the case of any preparation for external use, it is possible to exemplify the application in an appropriate amount to the disease portion once or several times a day. It is preferable that the treatment as described above is performed every day. In particular, in the case of the tinea unguium, luliconazole as the active ingredient, which is in an amount that cannot be brought about by any ordinary pharmaceutical preparation, can be transferred into the nail. Accordingly, the tinea unguium can be cured by means of only the external administration without taking any antifungal agent for a long period of time. Further, the recurrence and the reinfection cause great problems in relation to the tinea unguium. However, it is possible to avoid the recurrence and the reinfection as described above by administering the pharmaceutical composition of the present invention for 1 week to 2 weeks after the quietness of symptoms. In such a mode, the pharmaceutical composition of the present invention has the preventive effect.

### EXAMPLES

The present invention will be explained in further detail below as exemplified by Examples. However, the present invention is not limited to Examples described below.

### Example 1

Pharmaceutical preparations of luliconazole 1 to 4 were manufactured in accordance with the following formulations. That is, formulation components were solubilized by being heated and stirred, followed by being stirred and cooled to obtain Pharmaceutical preparations 1 to 4 each having an agent form of lotion. The preparations were stored at 60° C. for 3 weeks, and the content of the amide form was measured by HPLC after the storage. Results are shown in Table 1. Accordingly, it is understood that the formation amount of the amide form is increased depending on the type of polyhydric alcohol. Further, it is understood that Pharmaceutical preparation 4, in which the suppression is caused by the suppressing effect of diisopropyl adipate, is the pharmaceutical composition of the present invention.

HPLC condition: ODS-2 4.6×150 mm, column temperature: 40° C., mobile phase: 0.15% sodium undecane-1-sulfonate mixture liquid (water/acetonitrile/acetic acid (100) (50:49:1, v/v/v)) solution, flow rate: 1.0 mL/min., detection: 295 nm.

TABLE 1
(unit other than those described below: g)

| | Pharma- | Pharma- | Pharma- | Pharma- |
|---|---|---|---|---|
| | ceutical | ceutical | ceutical | ceutical |
| | prepa- | prepa- | prepa- | prepa- |
| | ration 1 | ration 2 | ration 3 | ration 4 |
| Luliconazole | 1 | 1 | 1 | 1 |
| 1,3-Butanediol | | balance | | |
| Macrogol 400 | | | balance | |
| (polyethylene glycol) | | | | |
| NMP | balance | | | |
| Diisopropyl adipate | | | | balance |
| | 100 ml | 100 ml | 100 ml | 100 ml |
| 60° C. for 3 weeks | | | | |
| (% by mass with respect | | | | |
| to luliconazole) | | | | |
| Amide form | 0.11 | 0.36 | 0.32 | 0 |

### Example 2

Pharmaceutical preparations 5 to 7 and Pharmaceutical preparation 36 were manufactured in the same manner as in Example 1. Results are shown in Table 2. It is understood that the amide form is suppressed by diisopropyl adipate.

TABLE 2

(unit other than those described below: g)

| | Pharma- | Pharma- | Pharma- | Pharma- |
|---|---|---|---|---|
| | ceutical | ceutical | ceutical | ceutical |
| | prepa- | prepa- | prepa- | prepa- |
| | ration 5 | ration 6 | ration 7 | ration 36 |
| Luliconazole | 1 | 1 | 1 | 1 |
| Crotamiton | 1 | 1 | 1 | 1 |
| Diisopropyl | 5 | 5 | 5 | |
| adipate | | | | |
| 1,3-Butanediol | 30 | | | 30 |
| Macrogol 400 | | 30 | | |
| Polypropylene | | | 20 | |
| glycol 2000 | | | | |
| Water | 30 | 30 | 30 | 30 |
| Anhydrous | 33 | 33 | 43 | 38 |
| ethanol | | | | |
| Total amount | 100 | 100 | 100 | 100 |
| Amide form after | 0.02 | 0.47 | 0.00 | 0.13 |
| storage at 60° C. | | | | |
| for 3 weeks (% | | | | |
| by mass with | | | | |
| respect to | | | | |
| luliconazole) | | | | |

### Example 3

Pharmaceutical preparations 8 to 11 and 37 were manufactured in the same manner as in Example 1, and the amount of the amide form was measured after the storage at 60° C. for 3 weeks. Results are shown in Table 3. Basically, it is understood that polyhydric alcohol and benzyl alcohol have the action or function to facilitate the formation of the amide form. It is understood that these pharmaceutical preparations are not the pharmaceutical composition of the present invention, because the amide form formation-facilitating component is contained in a large amount, and hence it is impossible to suppress the formation of the amide form.

TABLE 3
(unit other than those described below: g)

| | Pharma- | Pharma- | Pharma- | Pharma- | Pharma- |
|---|---|---|---|---|---|
| | ceutical | ceutical | ceutical | ceutical | ceutical |
| | prepa- | prepa- | prepa- | prepa- | prepa- |
| | ration 8 | ration 9 | ration 10 | ration 37 | ration 11 |
| Luliconazole | 1 | 1 | 1 | 1 | 1 |
| Benzyl alcohol | 4 | 4 | 4 | 3 | 4 |
| Diisopropyl | 5 | 5 | 5 | 5 | |
| adipate | | | | | |
| 1,3-Butanediol | 30 | | | 30 | |
| Macrogol 400 | | 30 | | | 30 |
| Polypropylene | | | 20 | | |
| glycol 2000 | | | | | |
| Water | 30 | 30 | 30 | 30.5 | 30 |
| Anhydrous | 30 | 30 | 40 | 30.5 | 35 |
| ethanol | | | | | |
| Total amount | 100 | 100 | 100 | 100 | 100 |
| Amide form after | 0.86 | 0.95 | 0.32 | 0.68 | 0.94 |
| storage at 60° C. | | | | | |
| for 3 weeks (% | | | | | |
| by mass with | | | | | |
| respect to | | | | | |
| luliconazole) | | | | | |

### Example 4

Pharmaceutical preparation 12 and Comparative pharmaceutical preparation 1 were manufactured in accordance with the following formulations in the same manner as in Example 1. The amide form contents of Pharmaceutical preparation 12 and Comparative pharmaceutical preparation 1, which were obtained after the storage at 60° C. for 3 weeks, were 0.02% by mass and 0.08% by mass respectively with respect to the luliconazole amount. It is understood that the formation of the amide form, which is facilitated by the addition of polyethylene glycol 400, is suppressed to be not more than 0.1% by mass, owing to the amide form formation-suppressing action of middle-chain fatty acid triglyceride and methyl ethyl ketone. Accordingly, it is understood that this pharmaceutical preparation is the pharmaceutical composition of the present invention.

TABLE 4
(% by mass)

| | | Comparative |
|---|---|---|
| | Pharmaceutical | pharmaceutical |
| Component | preparation 12 | preparation 1 |
| Luliconazole | 1 | 1 |
| Middle-chain fatty acid triglyceride | 10 | |
| Methyl ethyl ketone | 10 | 10 |
| Polyethylene glycol 400 | 20 | 20 |
| Anhydrous ethanol | balance | balance |
| Phosphoric acid | 0.1 | 0.1 |

### Example 5

Pharmaceutical preparations 13 to 16 and 38 to 40 were manufactured in accordance with the following formulations in the same manner as in Example 1. The amide form formation amounts, which were obtained after the storage at 60° C. for 3 weeks, were also measured. Results are shown in Table 5. Accordingly, it is considered that the amide form formation is suppressed by diisopropyl adipate. Further, it is also understood that pyrrolidones have such a tendency that the amide form formation is facilitated with ease. It is affirmed that only Pharmaceutical preparation 13 and Pharmaceutical preparation 39 of these pharmaceutical preparations are the pharmaceutical preparations of the present invention.

TABLE 5
(Unit other than those described below: g)

| | Pharma- | Pharma- | Pharma- | Pharma- | Pharma- | Pharma- | Pharma- |
|---|---|---|---|---|---|---|---|
| | ceutical | ceutical | ceutical | ceutical | ceutical | ceutical | ceutical |
| | prepa- | prepa- | prepa- | prepa- | prepa- | prepa- | prepa- |
| | ration 13 | ration 14 | ration 15 | ration 16 | ration 38 | ration 39 | ration 40 |
| Luliconazole | 1 | 1 | 1 | 1 | 1 | 1 | 1 |
| Crotamiton | 1 | | | 99 | | | |
| Benzyl alcohol | | 4 | | | 4 | 4 | 4 |
| N-Methyl-2-pyrrolidone | | | 8 | | | | |
| Methyl ethyl ketone | | | | | | 10 | |
| Diisopropyl adipate | 5 | 5 | 5 | | | 5 | 5 |
| 1,3-Butanediol | 30 | 30 | 30 | | 30 | 30 | 30 |
| Phosphoric acid | | | | | | | 0.1 |
| Water | 30 | 30 | 30 | | 30 | 30 | 30 |
| Anhydrous ethanol | 33 | 30 | 26 | | 35 | 20 | 29.9 |
| Total amount | 100 | 100 | 100 | 100 | 100 | 100 | 100 |
| Amide form after storage | 0.02 | 0.86 | 3.99 | 0.25 | 0.27 | 0.19 | 0.21 |
| at 60° C. for 3 weeks | | | | | | | |
| (% by mass with respect | | | | | | | |
| to luliconazole) | | | | | | | |

### Example 6

Pharmaceutical preparations 17 to 21 and 41 were manufactured in accordance with the following formulations in the same manner as in Example 1, and the amide form formation amounts were measured. Results are shown in Table 6. Accordingly, it is understood that the amide form formation-suppressing action is possessed by local anesthetic and antihistamine. Therefore, it can be also understood that the formation of the amide form is suppressed by allowing these components to coexist when any component, which facilitates the formation of the amide form, is contained. Further, it is also understood that Pharmaceutical preparations 20, 21, 41 are the preparations for external use of the present invention.

TABLE 6
(Unit other than those described below: [symbol] )

| | Pharma- | Pharma- | Pharma- | Pharma- | Pharma- | Pharma- |
|---|---|---|---|---|---|---|
| | ceutical | ceutical | ceutical | ceutical | ceutical | ceutical |
| | prepa- | prepa- | prepa- | prepa- | prepa- | prepa- |
| | ration 17 | ration 18 | ration 19 | ration 20 | ration 21 | ration 41 |
| Luliconazole | 1 | 1 | 1 | 1 | 1 | [symbol] |
| N-Methyl-2-pyrrolidone | 8 | 8 | 8 | 8 | 8 | [symbol] |
| Benzyl alcohol | | | | | | |
| Diisopropyl adipate | 5 | 5 | 5 | 5 | 5 | [symbol] |
| 1,3-Butanediol | 30 | 30 | 30 | 30 | 30 | 3 [symbol] |
| Lidocaine | | 1 | | | 1 | |
| Diphenhydramine hydrochloride | | | 1 | | 1 | |
| Chlorpheniramine maleate | | | | 1 | | |
| Water | 30 | 30 | 30 | 30 | 30 | 3 [symbol] |
| Anhydrous ethanol | 26 | 25 | 25 | 25 | 24 | 2 [symbol] |
| Total amount | 100 | 100 | 100 | 100 | 100 | 10 [symbol] |
| Amide form after storage | 0.49 | 0.28 | 0.30 | 0.10 | 0.11 | 0.0 [symbol] |
| at 60° C. for 3 weeks | | | | | | |
| (% by mass with respect | | | | | | |
| to luliconazole) | | | | | | |

[symbol]

indicates data missing or illegible when filed

### Example 7

Pharmaceutical preparations 22 to 30 and 42 to 44 were manufactured in accordance with the following formulations in the same manner as in Example 1, and the amide form formation amounts were measured. Results are shown in Table 7. It is understood that the formation of the amide form is suppressed for any one of Pharmaceutical preparations 22 to 29 as compared with Pharmaceutical preparation 30. That is, it is evident that the amide form formation-suppressing effect is acknowledged for phosphoric acid, lactic acid, citric acid, tartaric acid, middle-chain fatty acid triglyceride, triacetin, ethylene glycol salicylate, triethyl citrate, and diethylene glycol monoethyl ether. However, the value for the amide form after the storage at 60° C. for 3 weeks exceeds 0.2% by mass. Therefore, any one of Pharmaceutical preparations 22 to 30 and 42 does not belong to the pharmaceutical composition of the present invention. In other words, it is affirmed that the technical scope of the amide form formation-suppressing agent of the present invention is carried out in the case of these pharmaceutical preparations, but these pharmaceutical preparations do not belong to the technical scope of the pharmaceutical composition of the present invention. It is also affirmed that such a situation arises, because the amide form formation-facilitating agent is contained.

The amide form formation-suppressing effect is acknowledged in relation to Pharmaceutical preparations 43 and 44, and Pharmaceutical preparations 43 and 44 belong to the pharmaceutical composition of the present invention.

TABLE 7
(Unit other than those described below: g)

| | Pharma- | Pharma- | Pharma- | Pharma- | Pharma- | Pharma- |
|---|---|---|---|---|---|---|
| | ceutical | ceutical | ceutical | ceutical | ceutical | ceutical |
| | prepa- | prepa- | prepa- | prepa- | prepa- | prepa- |
| | ration 22 | ration 23 | ration 24 | ration 25 | ration 26 | ration 27 |
| Luliconazole | 1 | 1 | 1 | 1 | 1 | 1 |
| N-Methyl-2-pyrrolidone | 8 | 8 | 8 | 8 | 8 | 8 |
| Benzyl alcohol | | | | | | |
| Diisopropyl adipate | 5 | 5 | 5 | 5 | 5 | 5 |
| 1,3-Butylene glycol | 30 | 30 | 30 | 30 | 30 | 30 |
| Phosphoric acid | 0.1 | | | | | |
| Lactic acid | | 0.1 | | | | |
| Citric acid | | | 0.1 | | | |
| Tartaric acid | | | | 0.1 | | |
| Middle-chain fatty acid triglyceride | | | | | | |
| Triacetin | | | | | 5 | |
| Ethylene glycol salicylate | | | | | | 5 |
| Triethyl citrate | | | | | | |
| Diethylene glycol monoethyl ether | | | | | | |
| Water | 30 | 30 | 30 | 30 | 30 | 30 |
| Anhydrous ethanol | 25.9 | 25.9 | 25.9 | 25.9 | 21 | 21 |
| Total amount | 100 | 100 | 100 | 100 | 100 | 100 |
| Amide form after storage | 0.68 | 0.64 | 0.71 | 0.74 | 0.91 | 0.78 |
| at 60° for 3 weeks | | | | | | |
| (% by mass with respect | | | | | | |
| to luliconazole) | | | | | | |

(Unit other than those described below: g)

| | Pharma- | Pharma- | Pharma- | Pharma- | Pharma- | Pharma- |
|---|---|---|---|---|---|---|
| | ceutical | ceutical | ceutical | ceutical | ceutical | ceutical |
| | prepa- | prepa- | prepa- | prepa- | prepa- | prepa- |
| | ration 28 | ration 29 | ration 30 | ration 42 | ration 43 | ration 44 |
| Luliconazole | 1 | 1 | 1 | 1 | 1 | 1 |
| N-Methyl-2-pyrrolidone | 8 | 8 | 8 | 8 | | |
| Benzyl alcohol | | | | | 4 | 4 |
| Diisopropyl adipate | 5 | 5 | 5 | 5 | 5 | 5 |
| 1,3-Butylene glycol | 30 | 30 | 30 | 30 | 30 | 30 |
| Phosphoric acid | | | | | | |
| Lactic acid | | | | | 0.1 | |
| Citric acid | | | | | | |
| Tartaric acid | | | | | | |
| Middle-chain fatty acid triglyceride | | | | 0.1 | | |
| Triacetin | | | | | | |
| Ethylene glycol salicylate | | | | | | 5 |
| Triethyl citrate | 5 | | | | | |
| Diethylene glycol monoethyl ether | | 5 | | | | |
| Water | 30 | 30 | 30 | 30 | 30 | 30 |
| Anhydrous ethanol | 21 | 21 | 26 | 25.9 | 29.9 | 25 |
| Total amount | 100 | 100 | 100 | 100 | 100 | 100 |
| Amide form after storage | 0.94 | 1.30 | 3.99 | 0.68 | 0.06 | 0.05 |
| at 60° for 3 weeks | | | | | | |
| (% by mass with respect | | | | | | |
| to luliconazole) | | | | | | |

### Example 8

Pharmaceutical preparations 31 to 38, 46, and 47 were manufactured in accordance with the following formulations in the same manner as in Example 1, and the amide form formation amounts were measured. Results are shown in Table 8. The following fact is understood. That is, any one of Pharmaceutical preparations 31 to 38, 46, and 47 contains the component which facilitates the formation of the amide form and the component which suppresses the formation of the amide form, wherein the formation amount of the amide form is not more than 0.2% by mass under the storage condition at 60° C. for 3 weeks, and they are the pharmaceutical compositions of the present invention. That is, it has been proved that the formation of the amide form is suppressed by simultaneously containing the component which suppresses the formation of the amide form when the component, which facilitates the formation of the amide form, is contained in accordance with the method of the present invention.

TABLE 8
(Unit other than those described below: g)

| | Pharma- | Pharma- | Pharma- | Pharma- | Pharma- | Pharma- | Pharma- | Pharma- |
|---|---|---|---|---|---|---|---|---|
| | ceutical | ceutical | ceutical | ceutical | ceutical | ceutical | ceutical | ceutical |
| | prepa- | prepa- | prepa- | prepa- | prepa- | prepa- | prepa- | prepa- |
| | ration 45 | ration 31 | ration 32 | ration 33 | ration 34 | ration 35 | ration 46 | ration 47 |
| Luliconazole | 1 | 1 | 1 | 1 | 1 | 1 | 1 | 1 |
| Benzyl alcohol | 1 | 1 | 1 | 1 | 1 | 1 | 1 | 1 |
| N-Methyl-2-pyrrolidone | | 5 | | | | 5 | | |
| Isopropyl myristate | 5 | | 5 | 5 | 5 | | 5 | |
| Diisopropyl adipate | | | | | | | | 4 |
| Macrogol 400 | | | 8 | | 8 | | | |
| Propylene glycol | 5 | 5 | 5 | 5 | 5 | 5 | 5 | |
| Middle-chain fatty acid | 8 | 8 | | 8 | | 8 | 8 | |
| triglyceride | | | | | | | | |
| Cetostearyl alcohol | 6 | 6 | 6 | 6 | 6 | 6 | 6 | |
| White Vaseline | | | | | | | | 10 |
| Cetanol | | | | | | | | 5 |
| Synthetic squalane | | | | | | | | 2 |
| Polysorbate 60 | 3 | 3 | 3 | 3 | 3 | 3 | 3 | |
| Sorbitan monostearate | 0.75 | 0.75 | 0.75 | 0.75 | 0.75 | 0.75 | 0.75 | 0.5 |
| Glycerol monostearate | | | | | | | | 0.5 |
| Polyoxyethylene cetyl | | | | | | | | 3 |
| ether | | | | | | | | |
| Dibutylhydroxytoluene | 0.02 | 0.02 | 0.02 | | | 0.02 | 0.02 | |
| Methyl paraoxybenzoate | 0.14 | 0.14 | 0.14 | | | 0.14 | 0.14 | |
| Phosphoric acid | | | | | | 0.1 | | |
| 1-Menthol | | | | | | | 1 | 1 |
| Hydrocortisone acetate | | | | | | | 1 | 1 |
| Water | 70.09 | 70.09 | 70.09 | 70.25 | 70.25 | 69.99 | 68.09 | 71 |
| Total amount | 100 | 100 | 100 | 100 | 100 | 100 | 100 | 100 |
| Amide form after storage | 0.02 | 0.01 | 0.00 | 0.03 | 0.00 | 0.10 | 0.02 | 0.00 |
| at 60° for 3 weeks | | | | | | | | |
| (% by mass with respect | | | | | | | | |
| to luliconazole) | | | | | | | | |

Polysorbate: polyoxyethylene (20) sorbitan oleic acid ester

### INDUSTRIAL APPLICABILITY

The present invention is applicable to medicines.

1. A method for producing a pharmaceutical composition characterized in that
a content of an amide form represented by Chemical Formula (2) is not more than 0.2% by mass with respect to a charged amount of luliconazole after storage at 60° C. for 3 weeks or at 40° C. for 6 months,
the method comprising:
a step of performing under a severe condition (60° C. for 3 weeks) or an accelerated condition (40° C. for 6 months) as to a formulation candidate containing:
1) luliconazole represented by Chemical Formula (1); and
2-1) a combination of diisopropyl adipate; and one component or two or more components selected from 1,3-butanediol, propylene glycol, and polypropylene glycol, without comprising benzyl alcohol, or N-methyl-2-pyrrolidone; or 2-2) a combination of methyl ethyl ketone; and one component or two or more components selected from 1,3-butanediol, polyethylene glycol, propylene glycol, polypropylene glycol, and N-methyl-2-pyrrolidone;
measuring a content of an amide form represented by Chemical Formula (2) by HPLC utilizing a following HPLC condition, and
selecting a formulation of which content of an amide form is not more than 0.2% by mass as a pharmaceutical composition;
HPLC condition: ODS-2 4.6×150 mm, column temperature: 40° C., mobile phase: 0.15% sodium undecane-1-sulfonate mixture liquid (water/acetonitrile/acetic acid (100) (50:49:1, v/v/v)) solution, flow rate: 1.0 mL/min., detection: 295 nm:

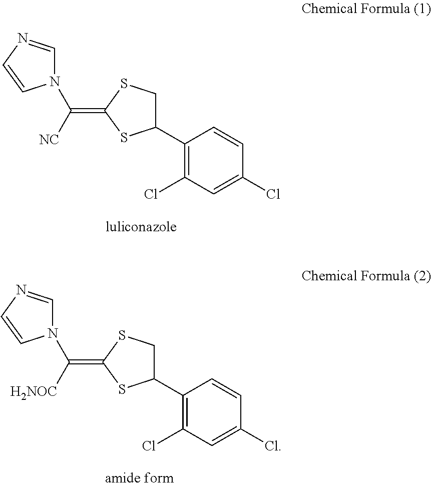 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

2.-6. (canceled)

7. The method for producing a pharmaceutical composition according to claim 1, wherein the pharmaceutical composition is a liquid agent or a cream agent.

8. A conversion-suppressing agent consisting of diisopropyl adipate for suppressing conversion of luliconazole represented by Chemical Formula (1) into an amide form represented by Chemical Formula (2) under a storage condition at 60° C. for 3 weeks or at 40° C. for 6 months, for a composition containing 1) luliconazole represented by Chemical Formula (1); and 2) one component or two or more components selected from 1,3-butanediol, propylene glycol, and polypropylene glycol, without comprising benzyl alcohol, or N-methyl-2-pyrrolidone:

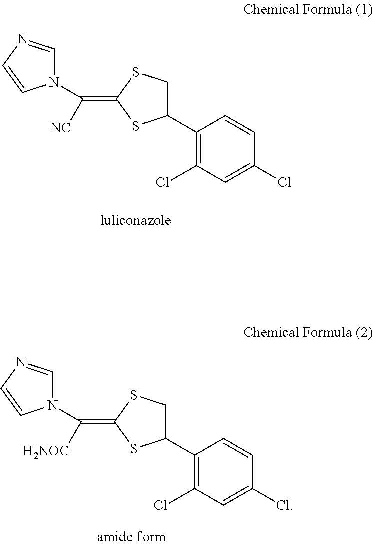 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]

9.-10. (canceled)

11. A conversion-suppressing agent consisting of methyl ethyl ketone for suppressing conversion of luliconazole represented by Chemical Formula (1) into an amide form represented by Chemical Formula (2) under a storage condition at 60° C. for 3 weeks or at 40° C. for 6 months, for a composition containing 1) luliconazole represented by Chemical Formula (1); and 2) one component or two or more components selected from 1,3-butanediol, polyethylene glycol, propylene glycol, polypropylene glycol, and N-methyl-2-pyrrolidone: 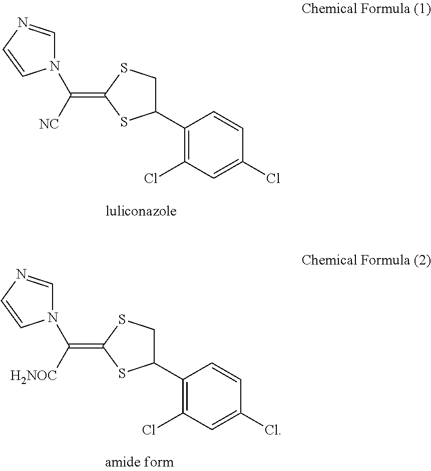 [SMILES: N#C/C(=C1/SCC(c2ccc(Cl)cc2Cl)S1)n1ccnc1 ; NC(=O)/C(=C1/SCC(c2ccc(Cl)cc2Cl)S1)n1ccnc1]
